(19) United States
(12) Patent Application Publication
(10) Pub. No.: US 2019/0054045 A1
(43) Pub. Date: Feb. 21, 2019

(54) METHODS AND COMPOSITIONS FOR TREATING SKIN CONDITIONS

(71) Applicant: Larissa Z. SCANLAN, Delray Beach, FL (US)

(72) Inventor: Larissa Z. SCANLAN, Delray Beach, FL (US)

(21) Appl. No.: 16/106,582

(22) Filed: Aug. 21, 2018

Related U.S. Application Data
(60) Provisional application No. 62/548,132, filed on Aug. 21, 2017.

(51) Int. Cl. A61K 31/155; A61K 31/495; A61K 31/195; A61K 9/00; A61P 17/02; A61P 17/00

(52) U.S. Cl. CPC A61K 31/155; A61K 31/495; A61K 31/195; A61P 17/00; A61K 9/0053; A61P 17/02; A61K 9/0014

(57) ABSTRACT

The disclosure provides methods and compositions for treating skin conditions. In particular, the disclosure provides compositions comprising metformin either alone or in combination with at least one additional active agent, and methods for treating skin conditions using such compositions.

### CROSS-REFERENCE TO RELATED APPLICATIONS

This application claims the benefit of U.S. Provisional Application No. 62/548,132, filed Aug. 21, 2017, the disclosure of which is explicitly incorporated by reference herein in its entirety.

### BACKGROUND

Tissue fibrosis is characterized by excessive deposition of collagen and other extracellular matrix components. It is the major histopathologic feature of a variety of diseases and it can affect a variety of organ systems. When skin is affected by exuberant fibrosis, just like in other organ systems, it can result in functional impairment. In addition, it can also result in cosmetic disfigurement and be symptomatic with pain or pruritus. Excessive dermal fibrosis can result from a variety of processes. It can be a result of autoimmune disorders, metabolic disorders, or be induced by drugs or chemicals. It can also be localized to only the skin as a result from trauma, such as burns, accidents, or surgical procedures. While rare, there is a wide spectrum of systemic fibrotic disorders affecting the skin, such as scleroderma, nephrogenic fibrosing dermopathy, mixed connective tissue disease, scleromyxedema, scleredema, and eosinophilic fasciitis. Hypertrophic scars and keloids are a localized form of exuberant dermal fibrosis and are much more common. Other localized forms of scaring or dermal fibrosis includes morphea (localized scleroderma) and conditions that result in hair loss from scaring like frontal fibrosing alopecia, lichen planopilaris, central centrifugal cicatricial alopecia, folliculitis decalvans, and other forms of scaring hair loss. Although the incidence of either systemic fibrotic diseases affecting the skin and the incidence of hypertrophic and keloid scarring is not known, the treatment of these cases is estimated to cost over $1.3 billion and $6.3 billion, respectively, based on 2013 claims tabulations of the US population alone (Lim et al., J. Am. Acad. Dermatol. 76(5): 958-72 (2017)).

Currently, the treatment of systemic skin fibrosis is the use of systemic immunosuppressive medications with an array of potentially toxic side effects, while the treatment of hypertrophic scars and keloids includes intralesional corticosteroids, lasers, silicone sheeting, compression garments, surgical revision, superficial radiation therapy, and other modalities with limited success. Thus, there is a need in the art to develop a systemic and localized therapy to ameliorate exuberant dermal fibrosis, regardless of the underlying pathophysiology. To address the need for ameliorating tissue fibrosis that would have minimal systemic side effects or toxicity in cases of systemic disease, and ease of use and affordability for local treatment, what is needed in the art are additional compositions and methods for the treatment of systemic skin fibrosis, hypertrophic scars and keloids, and other skin conditions.

### SUMMARY

The disclosure provides methods and compositions for treating skin conditions. In particular, the disclosure provides compositions comprising metformin either alone or in combination with at least one additional active agent, and methods for treating skin conditions using such compositions. The disclosure also provides compositions comprising metformin either alone or in combination with at least one additional active agent for oral or topical applications.

In one aspect, the disclosure provides a composition for treating a skin condition in a patient, wherein the composition comprises metformin. In certain embodiments, the concentration of metformin is between 25 mg/mL and 2,500 mg/mL.

In certain embodiments of this aspect, the composition further comprises at least one additional active agent selected from the group consisting of an antipruritic, an antidepressant, an antifibrinolytic, or a beta-blocker.

In some embodiments, the composition comprises an antipruritic and the antipruritic is selected from the group consisting of an antihistamine, a corticosteroid, a counterirritant, a local anesthetic, an opioid agonist, an antiemetic, and a gabapentinoid.

In some embodiments, the composition comprises an antidepressant, wherein the antidepressant is a tri-cyclic antidepressant selected from the group consisting of doxepin, amitriptyline, and mirtazapine.

In some embodiments, the antipruritic is an antihistamine and the antihistamine is selected from the group consisting of Acrivastine, Alimemazine, Astemizole, Azatadine, Bromodiphenhydramine, Brompheniramine, Carbinoxamine, Cetirizine, Chlorpheniramine, Cimetidine, Clemastine, Cyproheptadine, Desloratadine, Dexchlorpheniramine, Dextrobrompheniramine, Dimenhydrinate, Diphenhydramine, Doxylamine, Famotidine, Fexofenadine, Hydroxyzine, Lafutidine, Levocetirizine, Loratadine, Meclizine, Mizolastine, Nizatidine, Quetiapine, Pheniramine, Promethazine, Pyrilamine, Ranitidine, Roxatidine, Tripelennamine, and Triprolidine. In one embodiment, the antihistamine is levocetirizine. In certain embodiments, the concentration of the antihistamine is between 0.1 mg/mL and 50 mg/mL.

In some embodiments, the antipruritic is a corticosteroid and the corticosteroid is selected from the group consisting of bethamethasone, clobetasol, cortisone, dexamethasone, fludrocortisone, hydrocortisone, methylprednisolone, prednisone, prednisolone, and triamcinolone.

In some embodiments, the antipruritic is a counterirritant and the counterirritant is selected from the group consisting of camphor, capsaicin, capsicum, garlic, menthol (mint oil), methyl nicotinate, methyl salicylate, and trolamine salicylate.

In some embodiments, the antipruritic is a local anesthetic and the local anesthetic is selected from the group consisting of the group of amide or ester anesthetics, such as benzocaine, dibucaine, dyclonine, lidocaine, marcaine, and prilocaine.

In some embodiments, the antipruritic is an opioid agonist and the opioid agonist is selected from the group consisting of naloxone, naltrexone, nalmefene, diprenorphine, nalorphine, nalorphine dinicotinate, levallorphan, and samidorphan.

In some embodiments, the antipruritic is an antiemetic and the antiemetic is selected from the group consisting of aprepitant, casopitant, rolapitant, dolasetron, granisetron, ondansetron, tropisetron, palonosetron, mirtazapine, domperidone, olanzapine, droperidol, haloperidol, chlorpromazine, prochlorperazine, alizapride, prochlorperazine, and metoclopramide.

In some embodiments, the antipruritic is a gabapentinoid and the gabapentinoid is selected from the group consisting of gabapentin, pregabalin, phenibut, atagabalin, mirogabalin, and gabapentin enacarbil.

In some embodiments, the composition comprises an antifibrolytic, wherein the antifibrolytic is tranexamic acid.

In some embodiments, the composition comprises a beta-blocker, wherein the beta-blocker is selected from the group consisting of carvediol, propranolol, and timolol.

In some embodiments, the skin condition is selected from the group consisting of surgical defects, scars, surgical scars, hypertrophic scars, keloids, systemic skin fibrosis, scleroderma, nephrogenic fibrosing dermopathy, mixed connective tissue disease, scleromyxedema, scleredema, eosinophilic fasciitis, morphea (localized scleroderma), scaring hair loss disorders (including frontal fibrosing alopecia, lichen planopilaris, central centrifugal cicatricial alopecia, folliculitis decalvans), acne, rosacea, melasma, psoriasis, seborrheic dermatitis, and atopic dermatitis.

In certain embodiments, the composition is formulated to be administered topically, transdermally, orally, by subcutaneous injection, post laser skin ablation, or microneedling.

In another aspect, the disclosure provides a method for treating a skin condition in a patient comprising administering any one of the compositions disclosed herein.

In certain embodiments of this aspect, the skin condition is selected from the group consisting of surgical defect, scar, surgical scar, keloid, systemic skin fibrosis, scleroderma, nephrogenic fibrosing dermopathy, mixed connective tissue disease, scleromyxedema, scleredema, eosinophilic fasciitis, morphea (localized scleroderma), scaring hair loss disorders (including frontal fibrosing alopecia/lichen planopilaris, central centrifugal cicatricial alopecia, folliculitis decalvans, acne, rosacea, melasma, psoriasis, seborrheic dermatitis, and atopic dermatitis.

In some embodiments, the compositions as disclosed here can be administered: (a) topically, transdermally, orally, by subcutaneous injection, post laser skin ablation, or microneedling; (b) once per day, twice per day, three times per day, four times per day, five times per day, or more than 5 times per day; and/or (c) for 1 week to 12 months, or more than 12 months.

### BRIEF DESCRIPTION OF THE DRAWINGS

FIG. 1A shows the results of treatment of a burn scar with a topical administration of a composition 250 mg/mL metformin in petrolatum ointment. FIG. 1A shows a burn scar pre-treatment.

FIG. 1B shows the results of treatment of a burn scar with a topical administration of a composition 250 mg/mL metformin in petrolatum ointment. FIG. 1B shows the same scar following one-month of treatment with the topical metformin.

FIG. 2A shows the results of treatment of a surgical defect with a topical administration of a composition 250 mg/mL metformin in petrolatum ointment. FIG. 2A shows a surgical defect pre-treatment.

FIG. 2B shows the results of treatment of a surgical defect with a topical administration of a composition 250 mg/mL metformin in petrolatum ointment. FIG. 2B shows the same surgical defect following one-month of treatment with the topical metformin.

FIG. 3A shows the results of treatment of systemic skin fibrosis by oral administration of a composition comprising metformin and levocetirizine. FIG. 3A shows the systemic skin fibrosis pre-treatment.

FIG. 3B shows the results of treatment of systemic skin fibrosis by oral administration of a composition comprising metformin and levocetirizine. FIG. 3B shows the same systemic skin fibrosis following two-months of treatment by oral administration of a composition comprising metformin and levocetirizine.

FIG. 4A shows the results of treatment of a surgical scar by topical administration of a composition comprising metformin and levocetirizine. FIG. 4A shows the surgical scar pre-treatment.

FIG. 4B shows the results of treatment of a surgical scar by topical administration of a composition comprising metformin and levocetirizine. FIG. 4B shows the same surgical scar following one-month of treatment by topical administration of a composition comprising metformin and levocetirizine.

### DETAILED DESCRIPTION

The disclosure provides methods and compositions for treating skin conditions. In particular, the disclosure relates to a composition comprising metformin either alone or in combination with at least one other additional agent, and methods for treating skin conditions using such compositions.

In one aspect, the disclosure provides a composition for treating a skin condition in a patient, wherein the composition comprises metformin.

As used herein, the term “metformin” refers to an agent belonging to the biguanide class of antidiabetics with antihyperglycemic activity. Metformin is a biguanide hypoglycemic agent used in the treatment of non-insulin-dependent diabetes mellitus not responding to dietary modification. Metformin is available under different brand names, including, for example, Glucophage, Glucophage XR, Fortamet, Glumetza, and Riomet.

Metformin may be administered in an amount suitable for its intended use. In some embodiments, the composition comprises metformin in an amount of, for example, about 1.0 mg/mL to about 10 mg/mL, about 10 mg/mL to about 20 mg/mL, about 20 mg/mL to about 30 mg/mL, about 30 mg/mL to about 40 mg/mL, about 40 mg/mL to about 50 mg/mL, about 50 mg/mL to about 60 mg/mL, about 60 mg/mL to about 70 mg/mL, about 70 mg/mL to about 80 mg/mL, about 80 mg/mL to about 90 mg/mL, about 90 mg/mL to about 100 mg/mL, about 100 mg/mL to about 200 mg/mL, about 200 mg/mL to about 300 mg/mL, about 300 mg/mL to about 400 mg/mL, about 400 mg/mL to about 500 mg/mL, about 500 mg/mL to about 600 mg/mL, about 600 mg/mL to about 700 mg/mL, about 700 mg/mL to about 800 mg/mL, about 800 mg/mL to about 900 mg/mL, about 900 mg/mL to about 1000 mg/mL, about 1000 mg/mL to about 1500 mg/mL, about 1500 mg/mL to about 2000 mg/mL, or about 2000 mg/mL to about 2500 mg/mL. In an embodiment, the composition comprises 250 mg/mL of metformin. In another embodiment, the composition comprises 500 mg/mL of metformin.

In certain embodiments, the composition further comprises at least one additional active agent selected from the group consisting of an antipruritic, an antidepressant, an antifibrinolytic, or a beta-blocker. In some embodiments, a non-limiting example of the composition may comprise metformin in combination with an antipruritic and an antidepressant. In other embodiments, the composition may comprise, for example, metformin in combination with an antipruritic and an antifibrinolytic. In yet other embodiments, the composition may comprise, for example, metformin in combination with an antipruritic and a beta-blocker. In some embodiments, the composition may comprise, for example, metformin in combination with an antipruritic, an antifibrinolytic, and a beta-blocker. In certain embodiments, the composition may comprise, for example, metformin in combination with one, two, three, or more additional active agents as disclosed herein. For example, in a non-limiting example, the composition may comprise metformin in combination with one, two, three, or more antipruritic agents.

In some embodiments, the composition comprises an antipruritic and the antipruritic is selected from the group consisting of an antihistamine, a corticosteroid, a counterirritant, a local anesthetic, an opioid agonist, an antiemetic, and a gabapentinoid.

In certain embodiments, the antipruritic is an antihistamine. As used herein, the term “antihistamine” refers to a class of therapeutic compounds that inhibits the action of histamine via one or more histamine receptors. Typically, antihistamines include, but are not limited to, H1-receptor antagonists and/or H2-receptor antagonists, and can be used to treat coughing, a cold, and/or an allergic reaction. Examples of suitable antihistamines include, without limitation, Acrivastine, Alimemazine, Astemizole, Azatadine, Bromodiphenhydramine, Brompheniramine, Carbinoxamine, Cetirizine, Chlorpheniramine, Cimetidine, Clemastine, Cyproheptadine, Desloratadine, Dexchlorpheniramine, Dextrobrompheniramine, Dimenhydrinate, Diphenhydramine, Doxylamine, Famotidine, Fexofenadine, Hydroxyzine, Lafutidine, Levocetirizine, Loratadine, Meclizine, Mizolastine, Nizatidine, Quetiapine, Pheniramine, Promethazine, Pyrilamine, Ranitidine, Roxatidine, Tripelennamine, and Triprolidine. In one embodiment, the antihistamine is levocetirizine.

An antihistamine may be in an amount suitable for its intended use. In some embodiments, the composition comprising metformin further comprises an antihistamine in an amount of, for example, about 0.1 mg/mL, about 0.2 mg/mL, about 0.25 mg/mL, about 0.3 mg/mL, about 0.4 mg/mL, about 0.5 mg/mL, about 0.6 mg/mL, about 0.7 mg/mL, about 0.75 mg/mL, about 0.8 mg/mL, about 0.9 mg/mL, about 1 mg/mL, about 1.25 mg/mL, about 1.5 mg/mL, about 1.75 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, or about 30 mg/mL.

In yet other embodiments, the composition comprising metformin further comprises an antihistamine in an amount of, for example, about 0.1 mg/mL to about 1 mg/mL, about 0.1 mg/mL to about 2 mg/mL, about 0.1 mg/mL to about 3 mg/mL, about 0.1 mg/mL to about 4 mg/mL, about 0.1 mg/mL to about 5 mg/mL, about 0.1 mg/mL to about 6 mg/mL, about 0.1 mg/mL to about 7 mg/mL, about 0.1 mg/mL to about 8 mg/mL, about 0.1 mg/mL to about 9 mg/mL, about 0.1 mg/mL to about 10 mg/mL, about 0.25 mg/mL to about 1 mg/mL, about 0.25 mg/mL to about 2 mg/mL, about 0.25 mg/mL to about 3 mg/mL, about 0.25 mg/mL to about 4 mg/mL, about 0.25 mg/mL to about 5 mg/mL, about 0.25 mg/mL to about 6 mg/mL, about 0.25 mg/mL to about 7 mg/mL, about 0.25 mg/mL to about 7.5 mg/mL, about 0.25 mg/mL to about 8 mg/mL, about 0.25 mg/mL to about 9 mg/mL, about 0.25 mg/mL to about 10 mg/mL, about 0.5 mg/mL to about 1 mg/mL, about 0.5 mg/mL to about 2 mg/mL, about 0.5 mg/mL to about 3 mg/mL, about 0.5 mg/mL to about 4 mg/mL, about 0.5 mg/mL to about 5 mg/mL, about 0.5 mg/mL to about 6 mg/mL, about 0.5 mg/mL to about 7 mg/mL, about 0.5 mg/mL to about 8 mg/mL, about 0.5 mg/mL to about 9 mg/mL, about 0.5 mg/mL to about 10 mg/mL, about 0.75 mg/mL to about 1 mg/mL, about 0.75 mg/mL to about 2 mg/mL, about 0.75 mg/mL to about 3 mg/mL, about 0.75 mg/mL to about 4 mg/mL, about 0.75 mg/mL to about 5 mg/mL, about 0.75 mg/mL to about 6 mg/mL, about 0.75 mg/mL to about 7 mg/mL, about 0.75 mg/mL to about 8 mg/mL, about 0.75 mg/mL to about 9 mg/mL, about 0.75 mg/mL to about 10 mg/mL, about 1 mg/mL to about 5 mg/mL, about 1 mg/mL to about 10 mg/mL, about 1 mg/mL to about 15 mg/mL, about 1 mg/mL to about 20 mg/mL, about 1 mg/mL to about 25 mg/mL, about 1 mg/mL to about 30 mg/mL, about 5 mg/mL to about 10 mg/mL, about 5 mg/mL to about 15 mg/mL, about 5 mg/mL to about 20 mg/mL, about 5 mg/mL to about 25 mg/mL, about 5 mg/mL to about 30 mg/mL, about 10 mg/mL to about 20 mg/mL, or about 10 mg/mL to about 30 mg/mL.

In some embodiments, the composition comprises an antidepressant, wherein the antidepressant is a tri-cyclic antidepressant selected from the group consisting of doxepin, amitriptyline, and mirtazapine. In certain embodiments, the antidepressant (for example, amitriptyline) may be present in the compositions as disclosed herein in an amount ranging from about 1.0 mg to about 100 mg, from about 10 mg to about 100 mg, from about 20 mg to about 100 mg, from about 30 mg to about 100 mg, from about 40 mg to about 100 mg, from about 50 mg to about 100 mg, from about 60 mg to about 100 mg, from about 70 mg to about 100 mg, from about 80 mg to about 100 mg, or from about 90 mg to about 100 mg. Alternatively, the antidepressant may be present in the compositions as disclosed herein in an amount ranging from 1 mg to 100 mg, from 10 mg to 100 mg, from 20 mg to 100 mg, from 30 mg to 100 mg, from 40 mg to 100 mg, from 50 mg to 100 mg, from 60 mg to 100 mg, from 70 mg to 100 mg, from 80 mg to 100 mg, or from 90 mg to 100 mg. The antidepressant may also be present in the compositions as disclosed herein in or about the following amounts: 1 mg, 5 mg, 10 mg, 15 mg, 20 mg, 25 mg, 30 mg, 35 mg, 40 mg, 45 mg, 50 mg, 60 mg, 70 mg, 80 mg, 90 mg, or 100 mg.

In certain embodiments, the composition comprises an antipruritic wherein the antipruritic is a corticosteroid and the corticosteroid is selected from the group consisting of bethamethasone, clobetasol, cortisone, dexamethasone, fludrocortisone, hydrocortisone, methylprednisolone, prednisone, prednisolone, and triamcinolone. In certain embodiments, the corticosteroid (for example, clobetasol) may be present in the compositions as disclosed herein in concentrations ranging from about 0.05% to about 5%, from about 1% to about 5%, from about 2% to about 5%, from about 3% to about 4%, or from about 3% to about 5%. Alternatively, the corticosteroid may be present in the compositions as disclosed herein in concentrations ranging from 0.05% to 5%, from 1% to 5%, from 2% to 5%, from 3% to 4%, or from 3% to 5%. The corticosteroid may also be present in the compositions as disclosed herein in or about the following amounts: 0.05%, 0.1%, 1%, 2%, 3%, 4%, or 5%.

In other embodiments, the composition comprises an antipruritic wherein the antipruritic is a counterirritant and the counterirritant is selected from the group consisting of camphor, capsaicin, capsicum, garlic, menthol (mint oil), methyl nicotinate, methyl salicylate, and trolamine salicylate. In certain embodiments, the counterirritant (for example, menthol) may be present in the compositions described herein in concentrations ranging from about 0.1% to about 10%, from about 1% to about 10%, from about 2% to about 10%, or from about 4% to about 8%. Alternatively, the counterirritant may be present in the compositions as disclosed herein concentrations ranging from 0.1% to 10%, from 1% to 10%, from 2% to 10%, or from 4% to 8%. The counterirritant may also be present in the compositions as disclosed herein in or about the following amounts: 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10%.

In some embodiments, the composition comprises an antipruritic wherein the antipruritic is a local anesthetic and the local anesthetic is selected from the group consisting of benzocaine, dibucaine, dyclonine, lidocaine, and prilocaine. In certain embodiments, the local anesthetic (for example, lidocaine or prilocaine) may be present in the compositions as disclosed herein in concentrations ranging from about 0.1% to about 5%, from about 1% to about 5%, from about 2% to about 5%, from about 3% to about 4%, or from about 3% to about 5%. Alternatively, lidocaine or prilocaine may be present in the compositions as disclosed herein in concentrations ranging from 0.1% to 5%, from 1% to 5%, from 2% to 5%, from 3% to 4%, or from 3% to 5%. Lidocaine or prilocaine may also be present in the compositions as disclosed herein in or about the following amounts: 0.1%, 1%, 2%, 3%, 4%, or 5%.

In certain embodiments, the composition comprises an antipruritic, wherein the antipruritic is an opioid agonist and the opioid agonist is selected from the group consisting of naloxone, naltrexone, nalmefene, diprenorphine, nalorphine, nalorphine dinicotinate, levallorphan, and samidorphan. In certain embodiments, the opioid agonist (for example, naltrexone) may be present in the compositions as disclosed herein in concentrations ranging from about 0.1% to about 5%, from about 1% to about 5%, from about 2% to about 5%, from about 3% to about 4%, or from about 3% to about 5%. Alternatively, naltrexone may be present in the compositions as disclosed herein in concentrations ranging from 0.1% to 5%, from 1% to 5%, from 2% to 5%, from 3% to 4%, or from 3% to 5%. Naltrexone may also be present in the compositions as disclosed herein in or about the following amounts: 0.1%, 1%, 2%, 3%, 4%, or 5%.

In other embodiments, the composition comprises an antipruritic wherein the antipruritic is an antiemetic and the antiemetic is selected from the group consisting of aprepitant, casopitant, rolapitant, dolasetron, granisetron, ondansetron, tropisetron, palonosetron, mirtazapine, domperidone, olanzapine, droperidol, haloperidol, chlorpromazine, prochlorperazine, alizapride, prochlorperazine, and metoclopramide. In certain embodiments, the antiemetic (for example, aprepitant) may be present in the compositions described herein in concentrations ranging from about 1% to about 30%, from about 1% to about 15%, from about 1% to about 10%, or from about 4% to about 8%. Alternatively, aprepitant may be present in the compositions as disclosed herein in concentrations ranging from 1% to 30%, from 1% to 15%, from 1% to 10%, or from 4% to 8%. Aprepitant may also be present in the compositions as disclosed herein in or about the following amounts: 1%, 2%, 3%, 4%, 5%, 6%, 7%, 8%, 9%, 10% 11%, 12%, 13%, 14%, 15%, 16%, 17%, 18%, 19%, 20%, 21%, 22%, 23%, 24%, 25%, 26%, 27%, 28%, 29%, or 30%.

In some embodiments, the composition comprises an antipruritic wherein the antipruritic is a gabapentinoid and the gabapentinoid is selected from the group consisting of gabapentin, pregabalin, phenibut, atagabalin, mirogabalin, and gabapentin enacarbil. In certain embodiments, the gabapentinoid (for example, gabapentin) may be present in the compositions described herein in concentrations ranging from about 1% to about 30%, from about 1% to about 15%, from about 1% to about 10%, or from about 4% to about 8%. Alternatively, gabapentin may be present in the compositions of as disclosed herein in concentrations ranging from 1% to 30%, from 1% to 15%, from 1% to 10%, or from 4% to 8%. Gabapentin may also be present in the compositions as disclosed herein in or about the following amounts: 1%, 2%, 3%, 4%, 5%, 6%, 7%, 8%, 9%, 10% 11%, 12%, 13%, 14%, 15%, 16%, 17%, 18%, 19%, 20%, 21%, 22%, 23%, 24%, 25%, 26%, 27%, 28%, 29%, or 30%.

In some embodiments, the composition comprises an antifibrinolytic, wherein the antifibrinolytic is tranexamic acid. In certain embodiments, the antifibrinolytic (for example, tranexamic acid) may be present in the compositions described herein in concentrations ranging from about 1% to about 10%, from about 2% to about 10%, from about 5% to about 10%, or from about 4% to about 8%. Alternatively, the antifibrinolytic may be present in the compositions as disclosed herein in concentrations ranging from 1% to 10%, from 2% to 10%, from 5% to 10%, or from 4% to 8%. The antifibrinolytic may also be present in the compositions as disclosed herein in or about the following amounts: 1%, 1.5%, 2%, 3%, 4%, 5%, 6%, 7%, 8%, 9%, or 10%.

In certain embodiments, the composition comprises a beta-blocker, wherein the beta-blocker is selected from the group consisting of carvediol, propranolol, and timolol. In some embodiments, the beta-blocker (for example, timolol) may be present in the compositions described herein in concentrations ranging from about 0.1% to about 10%, from about 1% to about 10%, from about 2% to about 10%, or from about 4% to about 8%. Alternatively, the beta-blocker may be present in the compositions as disclosed herein in concentrations ranging from 0.1% to 10%, from 1% to 10%, from 2% to 10%, or from 4% to 8%. The beta-blocker may also be present in the compositions as disclosed herein in or about the following amounts: 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10%.

In certain embodiments, the composition is prepared in a dose that is therapeutically effective to treat a skin disorder as defined herein. In one embodiment, the composition includes a combined therapeutically effective amount of metformin and an antihistamine. In some embodiments, the dosage of metformin may be insufficient to treat a skin disorder in the absence of the antihistamine, but together they are therapeutically effective. In another embodiment, the metformin dose is therapeutically sufficient without co-administration of the antihistamine.

In an embodiment, the composition includes about 25, 50, 200, 250, 300, 400, 500, 600, 700, 800, 900, 1000, 1500, 2000, or 2500 mg/mL of metformin and about 0.5, 1, 2, 3, 4, 5, 6, 7, 8, 9, or 10 mg/mL levocetirizine. In another embodiment, 25, 50, 200, 250, 300, 400, 500, 600, 700, 800, 900, 1000, 1500, 2000, or 2500 mg of metformin and about 0.5, 1, 2, 3, 4, 5, 6, 7, 8, 9, or 10 mg levocetirizine. In yet another embodiment, the composition includes about 250 mg/mL metformin and about 2.5 mg/mL levocetirizine. In another embodiment, the composition includes about 250 mg metformin and about 2.5 levocetirizine. In another embodiment, the composition includes about 500 mg/mL metformin and about 10 mg/mL levocetirizine. In another embodiment, the composition includes about 500 mg metformin and about 10 mg levocetirizine. In yet another embodiment, the composition includes about 250 mg/mL metformin and about 2.5 mg/mL levocetirizine. In yet another embodiment, the composition includes about 250 mg metformin and about 10 mg levocetirizine.

Dosing frequency will depend upon the pharmacokinetic parameters of the composition being used. Typically, a clinician will administer the composition until a dosage is reached that achieves the desired effect. In some embodiments, the composition is administered about every 24, 23, 22, 21, 20, 19, 18, 17, 16, 15, 14, 13, 12, 11, 10, 9, 8, 7, 6, 5, 4, 3, 2, or 1 hour(s). In certain embodiments, the composition is administered about every 24, 12, 8, 6, or 4 hours. In yet another embodiment, the composition is administered every 8 or 12 hours. In some embodiments, the composition is administered 2, 3, 4, 5, or 6 times during at least one 24 hour period. In another embodiment, the composition is administered 2 or 3 times during at least one 24 hour period. In some embodiments, the composition is administered two times per day (BID), three times per day (TID), four times a day (QID), five times per day, or six times per day. In certain embodiments, the length of treatment can be less than 1 week to 12 months, or more, for localized disease. For example, the length of treatment can be from about 1 week to about 4 weeks, from about 2 weeks to about 6 weeks, from about 4 weeks to about 8 weeks, from about 1 month to about 3 months, from about 2 months to about 4 months, from about 3 months to about 6 months, from about 6 months to about 12 months, or more. In some embodiments, treatment duration can be until disease remission, for example, from about 1 month to about 24 months, or more. In certain embodiments, treatment duration can be indefinite, for example, in cases of systemic disease.

In some embodiments, administration of the composition includes simultaneous or approximately simultaneous administration of both metformin and the antihistamine, whether co-formulated or individually formulated. However, staggered administration is also contemplated, whereby at least one dose of an active agent is administered sequentially. For example, metformin may be administered once a day, while the antihistamine is administered twice a day.

The term “patient” as used herein includes human and animal subjects.

A “skin disorder” is any condition that would benefit from treatment using the compositions as disclosed herein. “Skin disorder” and “skin condition” are used interchangeably herein and include chronic and acute disorders or diseases, and can include, but are not limited to dermal fibrosis from systemic diseases such as scleroderma, nephrogenic fibrosing dermopathy, mixed connective tissue disease, scleromyxedema, scleredema, and eosinophilic fasciitis, and also localized conditions with dermal fibrosis such as morphea (localized scleroderma), hypertrophic scars and keloids and conditions that result in hair loss from scaring like frontal fibrosing alopecia, lichen planopilaris, central centrifugal cicatricial alopecia, folliculitis decalvans, and other forms of scaring hair loss, as well as, acne, rosacea, melasma, psoriasis, seborrheic dermatitis, and atopic dermatitis.

The terms “treatment” or “treat” as used herein refer to both therapeutic treatment and prophylactic or preventative measures. Those in need of treatment include those having the skin disorder as well as those prone to have the skin disorder or those in which the skin disorder is to be prevented. In certain embodiments, the composition is administered to a subject in need of treatment once per day, twice per day, three times per day, four times per day, or more. The appropriate dose, frequency, and duration can be modified to address the particular needs of a particular subject by taking into account factors including, but not limited to, the age, gender, weight, and health of the subject; the severity, extent (e.g., surface area of skin condition), and location of the skin condition. In some embodiments, the length of treatment can be less than 1 week to 12 months, or more, for localized disease. In certain embodiments, treatment duration can be indefinite, for example, in cases of systemic disease. In some embodiments, treatment duration can be until disease remission.

The terms “pharmaceutical composition” or “therapeutic composition” as used herein refer to a compound or composition capable of inducing a desired therapeutic effect when properly administered to a patient.

The pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants (see, e.g., REMINGTON'S PHARMACEUTICAL SCIENCES (18th Ed., A. R. Gennaro, ed., Mack Publishing Company 1990), and subsequent editions of the same, incorporated herein by reference for any purpose).

The terms “effective amount” and “therapeutically effective amount” when used in reference to a pharmaceutical composition comprising metformin and an antihistamine, refer to an amount or dosage sufficient to produce a desired therapeutic result. More specifically, a therapeutically effective amount is an amount of metformin and an antihistamine sufficient to inhibit, for some period of time, one or more of the clinically defined pathological processes associated with the skin condition being treated. The effective amount may vary depending on the specific metformin and an antihistamine dosages that are being used, and also depends on a variety of factors and conditions related to the patient being treated and the severity of the skin disorder. For example, if the metformin and an antihistamine composition to be administered in vivo, factors such as the age, weight, and health of the patient as well as dose response curves and toxicity data obtained in preclinical animal work would be among those factors considered. The determination of an effective amount or therapeutically effective amount of a given pharmaceutical composition is well within the ability of those skilled in the art.

The route of administration of the compositions as disclosed herein is in accord with known methods, e.g., orally; through injection by intravenous, intraperitoneal, intracerebral (intraparenchymal), intracerebroventricular, intramuscular, intraocular, intraarterial, intraportal, or intralesional routes; by sustained release systems; or by implantation devices. In certain embodiments, the routes of administration can include, but are not limited to, oral, topical, transdermal, intralesional, post-laser ablation, or microneedeling. In one embodiment, the route of administration is topical or transdermal.

In some embodiments, the compositions may be formulated as a topical composition for transdermal delivery. As used herein, “transdermal” or “topical” delivery relates to delivery of a drug by passage into and through the skin or mucosal tissue. The terms “transdermal”, “topical”, and “transmucosal” are used interchangeably unless specifically stated otherwise. Exemplary dosage forms include creams, lotions, gels, oils, or ointments, or any other topical forms known to those skilled in the art. In certain embodiments, the compositions for topical administration may further comprise additional ingredients including, pharmaceutically acceptable carriers, moisturizers, oils, fats, waxes, surfactants, thickening agents, antioxidants, viscosity stabilizers, chelating agents, buffers, preservatives, perfumes, dyestuffs, lower alkanols, humectants, emollients, dispersants, sunscreens such as radiation blocking compounds or UV-blockers, antibacterials, antifungals, disinfectants, vitamins, antibiotics, anti-acne agents, as well as other suitable materials that do not have a significant adverse effect on the activity of the topical composition. For example, a composition may comprise the following additional ingredients: vitamin E acetate, sodium metabisulfite, and butylated hydroxytoluene. Those skilled in the art will readily recognize additional ingredients that may be included in the compositions described herein. Exemplary pharmaceutical acceptable carriers that may be used in the compositions may include water, mixtures of water and water-miscible solvents such as lower alkanols or vegetable oils, and water-soluble ophthalmologically acceptable non-toxic polymers (for example, cellulose derivatives such as methylcellulose), glycerin, propylene glycol, methylparaben, alginates, glyceryl stearate, PEG-100 stearate, cetyl alcohol, propylparaben, butylparaben, sorbitols, polyethoxylated anhydrosorbitol monostearate (TWEEN™), white petrolatum (VASELINE™), triethanolamine, emu oil, aloe vera extract, lanolin, cocoa butter, Lipoderm® base, Lipoderm® ActiveMax base, VersaBase® gel, and the like. The pharmaceutically acceptable carrier may be present in the compositions as disclosed herein in concentrations ranging from about 55% to about 65% or in concentrations ranging from 55% to 65%. In one embodiment, the pharmaceutically acceptable carrier may be white petrolatum (VASELINE™). In yet another embodiment, the pharmaceutically acceptable carrier may be Lipoderm® base. In yet another embodiment, the pharmaceutically acceptable carrier may be VersaBase® gel. Other ingredient lists with additional, fewer, or alternate ingredients may be used.

### EXAMPLES

The Examples that follow are illustrative of specific embodiments of the disclosure as provided herein, and various uses thereof. They are set forth for explanatory purposes only, and should not be construed as limiting the scope of the disclosure as provided herein in any way.

### Examples

### Example 1

### Topical Metformin to Treat a Burn Scar on Intact Skin

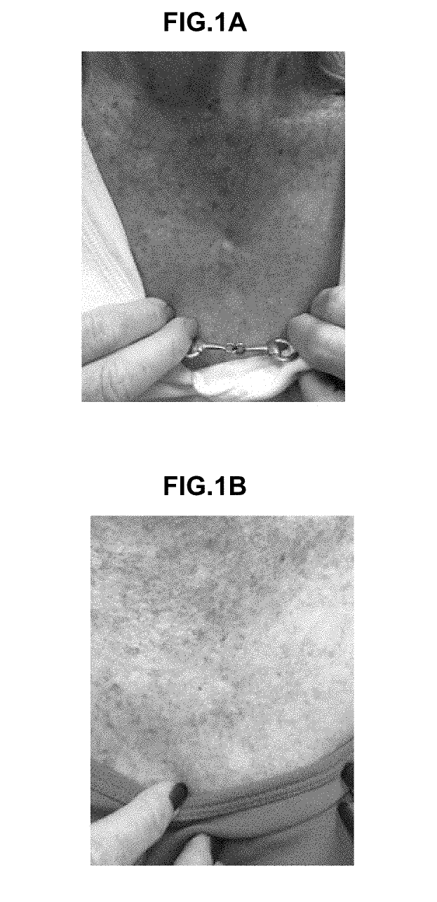

A patient with a burn scar from a previously treated skin cancer complaining of a raised red and itchy scar on her chest received topical metformin in petrolatum ointment at 250 mg/mL with directions to apply the lotion to the affected area (red, raised fibrotic scarred skin on chest) two times per day. Within 14 days, the scarring of the skin was visibly reduced with less redness, itch, and increased softness, and further improved after one month (observation by physician and patient). See FIG. 1.

### Example 2

### Topical Metformin was Tested to Treat Scarring on Open Skin

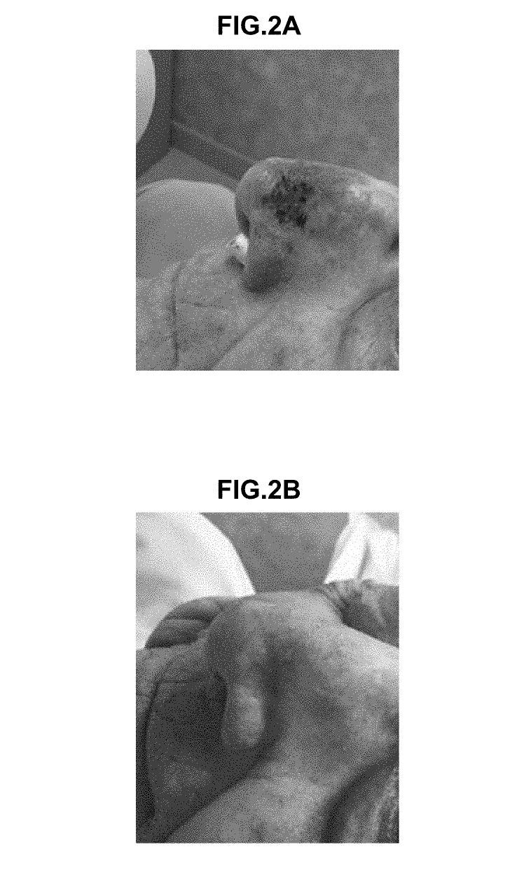

A patient with a previous surgical scar from removal of skin cancer with a scalpel received topical metformin ointment at 250 mg/mL with directions to apply the ointment twice a day to the surgical defect until healed (approximately 2-4 weeks). Within 4 weeks of application, the patient returned to clinic with minimal scaring and redness of area in comparison to previously observed patients healing with the use of topical petrolatum alone (control) (observation by physician). See FIG. 2.

### Example 3

### Oral Metformin and Levocetirizine to Treat Eosinophilic Fasciitis

Thirty-four year old female presented with eosinophilic fasciitis diagnosed a year ago that presented soon after childbirth.

History of present illness: Skin fibrosis began over her abdomen and rapidly spread to her arms, legs, chest, and back. Patient reported that her skin feels bound-down and has sensations of burning and pruritus.

Past medical history/Surgical history: Ewing's sarcoma of rib diagnosed at age 7 treated with chemotherapy and radiation. Renal cell carcinoma as an incidental finding with affected kidney removed without sequelae.

Social History: No alcohol, tobacco, or recreational drugs.

Current medications: Prednisone 10 mg/day, methotrexate 15 mg/day, sirolimus 2 mg/day.

Review of systems: non-contributory.

Physical Exam: Significant for firm, bound down and erythematous skin involving mainly arms and legs bilaterally.

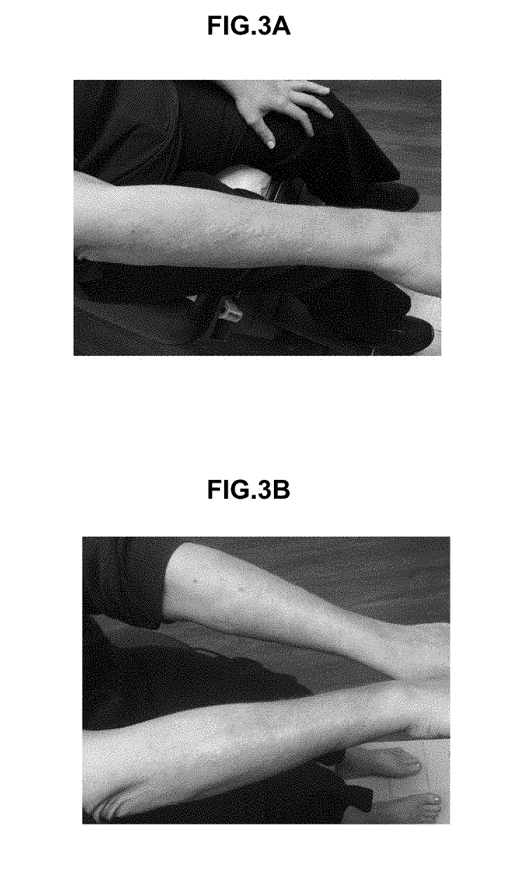

The patient was offered a trial of topical metformin 250 mg/mLe and levocetirizine 2.5 mg/mL in an ointment to affected areas to use twice a day along with her oral medications and told to return to clinic in one month. When the patient returned, she said her itch seemed much better while she was on the topical metformin and levocetirizine composition, but ran out after two weeks. Due to large involvement of body surface area, the patient was offered oral metformin at 500 mg/twice a day and oral levocetirizine 5 mg/daily. When the patient returned to clinic one month later, her skin was visibly improved (see FIG. 3). On a scale of 1 to 10, with 10 being the worst her skin felt, and a 1 being her normal skin, she said that before starting her treatment a year ago, it was a 10. After a year of oral medications, it went down to a 5 and was stable without any change for past 6 months. After just one month of the combination of metformin and levocetirizine, she is now a 2 on a severity scale of 1 to 10. After being stable on the metformin and levocetirizine oral combination, she has been able to wean off of the prednisone and is now taking methotrexate 10 mg/day with same amount of sirolimus. The goal is to completely wean her off of methotrexate and eventually the sirolimus as long as her disease continues to improve. See FIG. 3.

### Example 4

### Topical Composition Comprising Metformin and Levocetirizine was Tested to Treat a Post-Surgical Scar

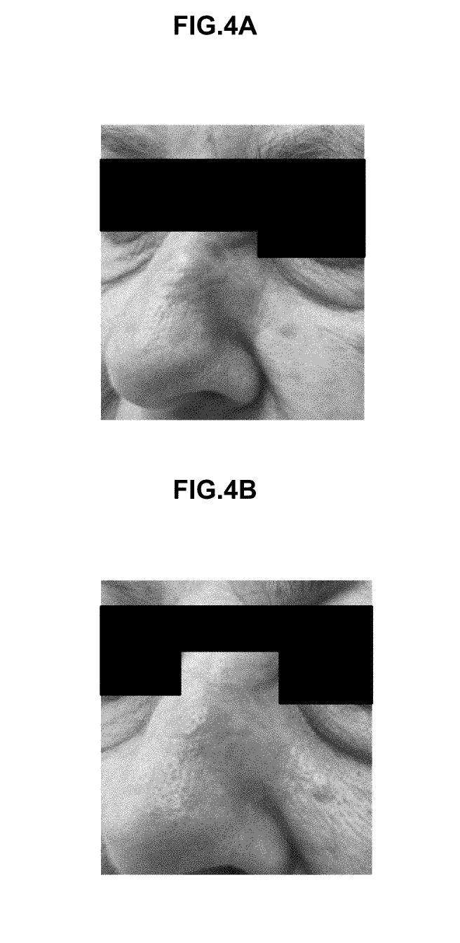

After surgical reconstruction of a skin cancer, patient presented with raised scar on her nose 6 weeks after surgery and complaining of “tightness.” She was given a combination of metformin 250 mg/mL with levocetirizine 2.5 mg/mL and told to apply it to her wound twice a day. One month later she presented with visibly decreased scaring and discomfort. See FIG. 4.

All references cited in this application are expressly incorporated by reference herein.

1. A composition for treating a skin condition in a patient, wherein the composition comprises metformin.

2. The composition of claim 1, wherein the composition further comprises at least one additional active agent selected from the group consisting of an antipruritic, an antidepressant, an antifibrinolytic, or a beta-blocker.

3. The composition of claim 2, wherein the composition comprises an antipruritic and the antipruritic is selected from the group consisting of an antihistamine, a corticosteroid, a counterirritant, a local anesthetic, an opioid agonist, an antiemetic, and a gabapentinoid.

4. The composition of claim 2, wherein the composition comprises an antidepressant, wherein the antidepressant is a tri-cyclic antidepressant selected from the group consisting of doxepin, amitriptyline, and mirtazapine.

5. The composition of claim 3, wherein the antipruritic is an antihistamine and the antihistamine is selected from the group consisting of Acrivastine, Alimemazine, Astemizole, Azatadine, Bromodiphenhydramine, Brompheniramine, Carbinoxamine, Cetirizine, Chlorpheniramine, Cimetidine, Clemastine, Cyproheptadine, Desloratadine, Dexchlorpheniramine, Dextrobrompheniramine, Dimenhydrinate, Diphenhydramine, Doxylamine, Famotidine, Fexofenadine, Hydroxyzine, Lafutidine, Levocetirizine, Loratadine, Meclizine, Mizolastine, Nizatidine, Quetiapine, Pheniramine, Promethazine, Pyrilamine, Ranitidine, Roxatidine, Tripelennamine, and Triprolidine.

6. The composition of claim 5, wherein the antihistamine is levocetirizine.

7. The composition of claim 3, wherein the antipruritic is a corticosteroid and the corticosteroid is selected from the group consisting of bethamethasone, clobetasol, cortisone, dexamethasone, fludrocortisone, hydrocortisone, methylprednisolone, prednisone, prednisolone, and triamcinolone.

8. The composition of claim 3, wherein the antipruritic is a counterirritant and the counterirritant is selected from the group consisting of camphor, capsaicin, capsicum, garlic, menthol (mint oil), methyl nicotinate, methyl salicylate, and trolamine salicylate.

9. The composition of claim 3, wherein the antipruritic is a local anesthetic and the local anesthetic is selected from the group consisting of benzocaine, dibucaine, dyclonine, lidocaine, and prilocaine.

10. The composition of claim 3, wherein the antipruritic is an opioid agonist and the opioid agonist is selected from the group consisting of naloxone, naltrexone, nalmefene, diprenorphine, nalorphine, nalorphine dinicotinate, levallorphan, and samidorphan.

11. The composition of claim 3, wherein the antipruritic is an antiemetic and the antiemetic is selected from the group consisting of aprepitant, casopitant, rolapitant, dolasetron, granisetron, ondansetron, tropisetron, palonosetron, mirtazapine, domperidone, olanzapine, droperidol, haloperidol, chlorpromazine, prochlorperazine, alizapride, prochlorperazine, and metoclopramide.

12. The composition of claim 3, wherein the antipruritic is a gabapentinoid and the gabapentinoid is selected from the group consisting of gabapentin, pregabalin, phenibut, atagabalin, mirogabalin, and gabapentin enacarbil.

13. The composition of claim 2, wherein the composition comprises an antifibrolytic, wherein the antifibrolytic is tranexamic acid.

14. The composition of claim 2, wherein the composition comprises a beta-blocker, wherein the beta-blocker is selected from the group consisting of carvediol, propranolol, and timolol.

15. The composition of claim 1, wherein the concentration of metformin is between 25 mg/mL and 2,500 mg/mL.

16. The composition of claim 5, wherein the concentration of the antihistamine is between 0.1 mg/mL and 50 mg/mL.

17. The composition of claim 1, wherein the skin condition is selected from the group consisting of surgical defects, scars, surgical scars, hypertrophic scars, keloids, systemic skin fibrosis, scleroderma, nephrogenic fibrosing dermopathy, mixed connective tissue disease, scleromyxedema, scleredema, eosinophilic fasciitis, morphea (localized scleroderma), scaring hair loss disorders (including frontal fibrosing alopecia, lichen planopilaris, central centrifugal cicatricial alopecia, folliculitis decalvans), acne, rosacea, melasma, psoriasis, seborrheic dermatitis, and atopic dermatitis.

18. The composition of claim 1, wherein the composition is formulated to be administered topically, transdermally, orally, by subcutaneous injection, post laser skin ablation, or microneedling.

19. A method for treating a skin condition in a patient comprising administering the composition of claim 1.

20. The method of claim 19, wherein the skin condition is selected from the group consisting of surgical defect, scar, surgical scar, keloid, systemic skin fibrosis, scleroderma, nephrogenic fibrosing dermopathy, mixed connective tissue disease, scleromyxedema, scleredema, eosinophilic fasciitis, morphea (localized scleroderma), scaring hair loss disorders (including frontal fibrosing alopecia/lichen planopilaris, central centrifugal cicatricial alopecia, folliculitis decalvans, acne, rosacea, melasma, psoriasis, seborrheic dermatitis, and atopic dermatitis.

21. The method of claim 19, wherein the composition is administered:
(a) topically, transdermally, orally, by subcutaneous injection, post laser skin ablation, or microneedling;
(b) once per day, twice per day, three times per day, four times per day, five times per day, or more than 5 times per day; and/or
(c) for 1 week to 12 months, or more than 12 months.
